(19) United States
(12) Patent Application Publication
(10) Pub. No.: US 2004/0250350 A1
(43) Pub. Date: Dec. 16, 2004

(54) Device for correction of sacral dysfuctions and simultaneous reduction of lumbar lordosis

(76) Inventor: Ronald Lee Leonard, Omaha, NE (US)

Correspondence Address: RONALD L. LEONARD, 13518 GRISSOM ST., OMAHA, NE, 68138, US

(21) Appl. No.: 10/827,723

(22) Filed: Apr. 19, 2004

Related U.S. Application Data
(60) Provisional application No. 60/464,040, filed on Apr. 21, 2003.

(51) Int. Cl. A61F 2/30

(52) U.S. Cl. 5/630

(57) ABSTRACT

A therapeutic devise consisting of a wedge shaped base with sloping quadrilateral elevation contacting the sacrum and overlapping the iliac bones at the sacroiliac joint. The top surface of the base is concave bilaterally to comfortably accommodate the buttocks of the user. A sacral attachment is placed on top of the central quadrilateral elevation of the base, itself consisting of four smaller rhomboid elevations that contact each inferior lateral angle of the sacrum and overlapping each sacroiliac joint near the sacral base. The overlapping of the sacroiliac joint acts to bias the sacrum to remain square within the pelvis. The overall slope of the base is maintained in the sacral attachment and acts to simultaneously reduce lumbar lordosis. The combination of decreased lumbar lordosis and squaring the sacrum within the pelvis allows relief of pain and discomfort from muscle strain of the lower back, lumbar intervertebral disc compression, lumbar and sacral nerve irritation, and many sacral dysfunctions simultaneously with one device.

### FIELD OF THE INVENTION

The invention relates to the field of therapeutic devices, specifically to a device to reduce lumbar lordosis and simultaneously correct sacral dysfunctions.

### BACKGROUND OF THE INVENTION

Many people suffer from low back, buttock, hip or leg pain. There exist many causes for this pain, the most common of which relate to spinal and sacral dysfunctions, or misalignment, affecting the muscles and nerve roots of the lower back and sacrum. Previous devices to treat low back pain have primarily dealt with either support or realignment of the lumbar spine or reduction of lumbar lordosis while ignoring the sacral dysfunctions such as sacral flexion, extension, rotation, and torsion, which often contribute to or are solely responsible for the pain. Many nerve roots enter and exit the sacrum and misalignment can impinge upon these nerves causing pain in the low back, buttock, hip or lower extremities. Sacral dysfunctions also put unnecessary strain on the lumbosacral, superior sacral articular, and sacroiliac joints causing discomfort. Each of the sacral dysfunctions will be further explained in the following drawings and discussion.

Presented here is a device designed to correct sacral dysfunctions while simultaneously reducing lumbar lordosis achieving a more thorough treatment of low back, buttock, hip or leg pain.

### DISCUSSION OF RELATED KNOWN ART

U.S. Pat. No. Des. 410,744 to Banister discloses a design relating to lumbar support. U.S. Pat. No. D469,648S to Meldeau discloses a design relating to lumbar support. U.S. Pat. No. 2002/0124318A1 to Loomos discloses a cushion to be used in a seated position to increase or reversed to decrease lumbar lordosis. The cushion also relates to alleviating pain from hemorrhoids and pressure ulcers.

U.S. Pat. No. 4,483,329 to Shamos discloses a device to bias the apex of a supine patient to reduce lumbar lordosis.

U.S. Pat. No. 5,201,761 to Serola discloses a dual use pillow for reducing lumbar lordosis of a supine patient and provide lumbar support to a seated patient.

U.S. Pat. No. 5,987,675 to Kim discloses a spinal support and stretch pillow system used in the supine or lateral-lying position. The device includes an adjustable cervical, thoracic, and lumbar system.

U.S. Pat. No. 6,132,004 to Carlino discloses a spinal support pillow to be used while seated.

### SUMMARY OF THE INVENTION

In one embodiment, this invention consists of a generally wedged shaped base with a central quadrilateral elevation projecting up from the top surface. The elevation maintains the same general slope of the base and is of a size that will accommodate an average sized human sacrum, overlapping the sacroiliac joints and contacting the iliac bones near the sacral base. The remainder of the top surface of the base is bilaterally concave to accommodate the buttocks of the user.

The slope of the elevation and wedge shaped base act to move the apex of the sacrum anteriorly when the invention is positioned appropriately beneath the pelvis of a supine user. The anterior movement of the sacral apex results in a decrease in lumbar lordosis and a gentle stretching of the erector spinae muscles, reducing spinal and muscular strain in the low back.

In another embodiment of the invention, a sacral attachment is placed over the central quadrilateral elevation of the base. The sacral attachment maintains the overall slope of the base and consists of four rhomboid elevations arranged to contact the sacrum on all four corners.

Specifically, two of the elevations contact and overlap the sacroiliac joints near the sacral base. Two elevations contact the apex of the sacrum at the inferior lateral angles. The placement of the four elevations acts to square the sacrum within the pelvis and to the pelvis, reducing sacral rotation and torsion. By maintaining the slope of the wedge shaped base, this embodiment also acts to reduce lumbar lordosis simultaneously as described in the first embodiment.

### BRIEF DESCRIPTION OF THE DRAWINGS

Any dimensions shown in drawings are solely to demonstrate proportions appropriate for use on an average sized human and it should be obvious that there is a great variation in human size and with that the need for variations in actual size of the invention.

FIG. 1 is a perspective view of the main base plate.

FIG. 2 is a perspective view of the quadrilateral rubber pad.

FIG. 3 is a perspective view of the sacral attachment.

FIG. 4 shows perspective views of the small (22) and large (23) rhomboid rubber comfort pads. Two each of these are needed.

FIG. 5 is a perspective view of the base plate with quadrilateral rubber pad, sacral attachment, and small and large rubber comfort pads in place and represents the fully assembled device.

FIG. 6 shows a top view of the base plate with quadrilateral rubber pad in place.

FIG. 7 is a front view of the base plate with quadrilateral rubber pad in place.

FIG. 8 is a side view of base plate with quadrilateral rubber pad in place.

FIG. 9 is a top view of the sacral attachment with the four rhomboid rubber comfort pads in place.

FIG. 10 is a front view of the sacral attachment with the four rhomboid rubber comfort pads in place.

FIG. 11 is a side view of the sacral attachment with the four rhomboid rubber comfort pads in place.

FIG. 12 is a front (anterior) view of the human sacrum.

FIG. 13 is a back (posterior) view of the human sacrum.

FIG. 14 shows a representation of the human sacrum with the possible axes of rotation 29, flexion and extension 28, and torsion 30A, 30B demonstrated by crossing lines.

### DETAILED DESCRIPTION OF THE INVENTION

FIG. 1 shows the main base plate of the present invention and consists of a generally wedge shaped box, the top surface of which slants downward from the back edge 2B of the device to the front rounded edge 2C. The preferred construction would be of a molded plastic, but any comparable material known to those skilled in the art such as molded rubber could be utilized. The base plate could be braced from within for strength in any number of manners obvious to those skilled in the art. On the top surface there is a quadrilateral shaped elevation 1A with the base nearest the front 1B being wider side-to-side than the base nearest the back 1C. The overall dimensions of the elevation should be comparable to that of a human sacrum. The quadrilateral elevation 1A has the same general downward slope from back 1C to front 1B as the top surface of the base plate. In each corner of the quadrilateral elevation 1A there are cylindrical holes 3,4,5,6 extending from the top surface 1A through the entire depth of the base plate. A flat surface 2E extends from the front edge 1B of the quadrilateral elevation 1A to the front rounded edge 2C of the base plate with a width equal to that of the front edge 1B of the quadrilateral elevation 1A and at the same overall slope as the side edges and top surface of the base plate. A flat quadrilateral shaped surface 2D extends upward from the back edge 1C of the quadrilateral elevation 1A to the back edge of the base plate at the same overall slope as the side edges and top surface of the base plate. The flat quadrilateral surface 2D is the same width side to side as the back edge 1C of the quadrilateral elevation 1A at the point it abuts the quadrilateral elevation 1A and narrows as it extends upward to the back edge of the base plate in a manner equal to that of the side edges of the quadrilateral elevation 1A.

On either side of the quadrilateral elevation 1A, flat surface 2E, and flat surface 2D the top surface forms a rounded indention 2A which arcs downward and then back up to meet the side edges of the base plate. The overall back to front slope of the indentions is equal to that of the side edges of the base plate.

Located on each side panel of the base plate there are indented handles 7. The handles 7 are located forward of a midpoint between the front rounded edge 2C and the back edge 2B.

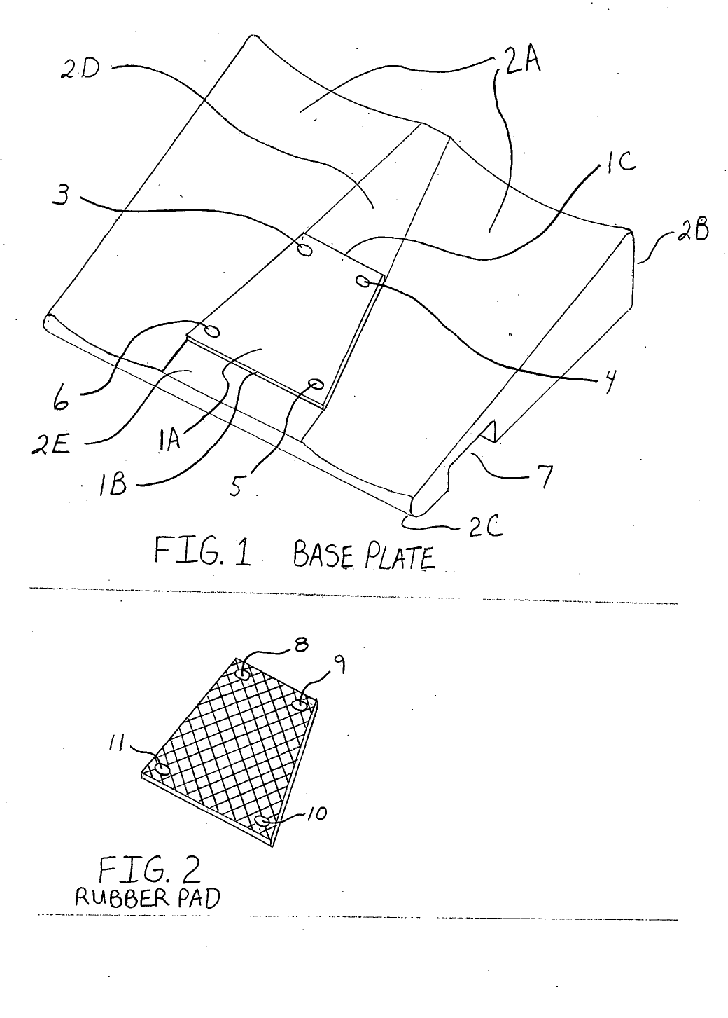

Shown in FIG. 2 is the quadrilateral rubber pad, which is of the same dimensions as the quadrilateral elevation 1A. In each corner of the quadrilateral rubber pad there are cylindrical holes 8,9,10,11 extending through the entire thickness of the pads. The holes are located in a position so that when the rubber pad is placed on top of the quadrilateral elevation 1A that each corresponds to the holes in the quadrilateral elevation 1A in the following manner 8-3,94,10-5,11-6.

The sacral attachment shown in FIG. 3 would be constructed from molded plastic or similar suitable material to attain a rigid configuration. The sacral attachment has an overall quadrilateral shape with the front edge 16A being wider side-to-side than the back edge 17A. A raised plateau is formed along the front edge, which forms two rhomboid elevations 14,15. Between the rhomboid elevations 14,15 there is a curved depression 16B. The curved depression 16B is wider side-to-side along the front edge 16A than along its back edge 16C. A similar elevated plateau is located along the back edge 17A of the sacral attachment, also forming two rhomboid elevations 12,13 in each back corner. There is a curved depression 17B between rhomboid elevations 12,13 which is wider along its front edge 17C than its back edge 17A. Projecting from the bottom surface of the sacral attachment are four cylindrical pegs 18,19,20,21. The cylindrical pegs 18,19,20,21 are located near each corner of the sacral attachment in a position so that when placed on top of the quadrilateral elevation 1A each peg fits into a corresponding cylindrical hole in both the rubber pad and quadrilateral elevation as follows 18-11,6; 19-10,5; 20-8,3; 21-9,4.

FIG. 4 shows the small 22 and large 23 rhomboid rubber comfort pads. There are two each of these required. Small pads 22 are the same dimensions as the back rhomboid elevations 12,13 and are to be placed or adhesed on top of the back rhomboid elevations 12,13. Large pads 23 are the same dimensions as the front rhomboid elevations 14,15 and are to be placed or adhesed on top of the front rhomboid elevations 14,15.

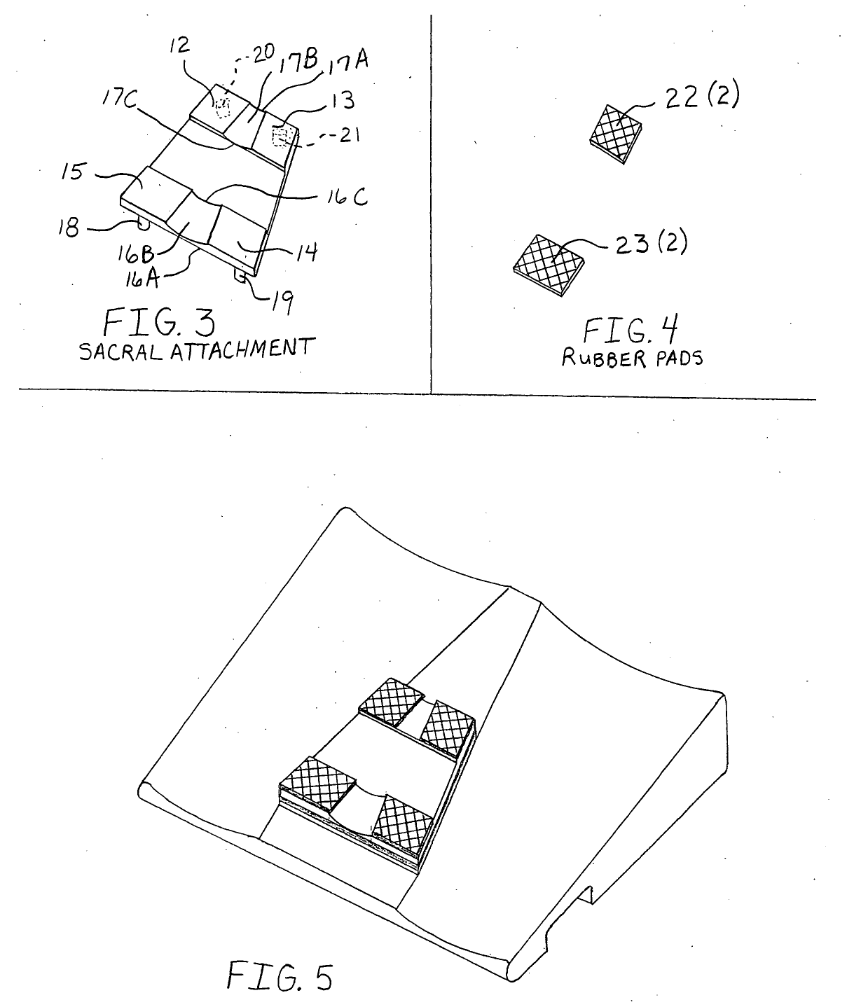

The completely assembled device is shown in FIG. 5 demonstrating the quadrilateral rubber pad (FIG. 2) in place or adhesed on quadrilateral elevation 1A of base plate (FIG. 1) with sacral attachment (FIG. 3) in place complete with rhomboid rubber comfort pads 22,23 in place or adhesed.

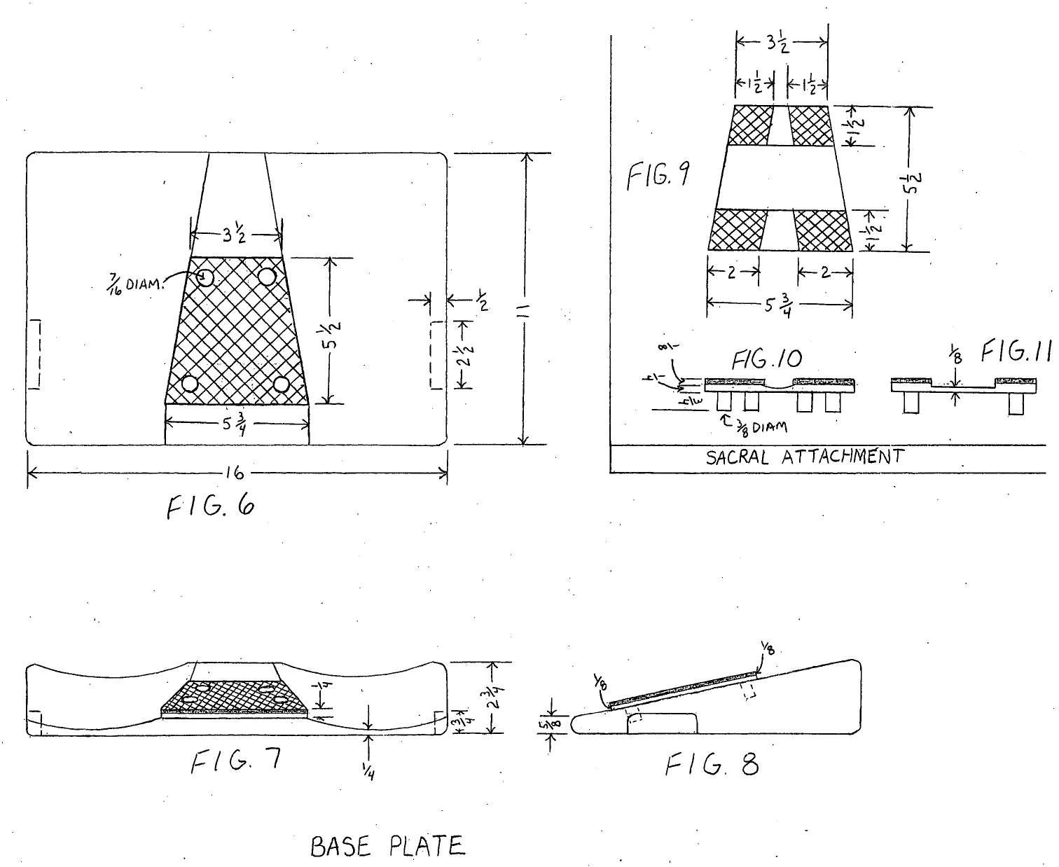

The dimensions shown in FIGS. 6 through 11 relate to an average size human sacrum and are shown only to demonstrate an average proportion between the different components. Please refer to the Brief Description of the Drawings for orientation on these drawings.

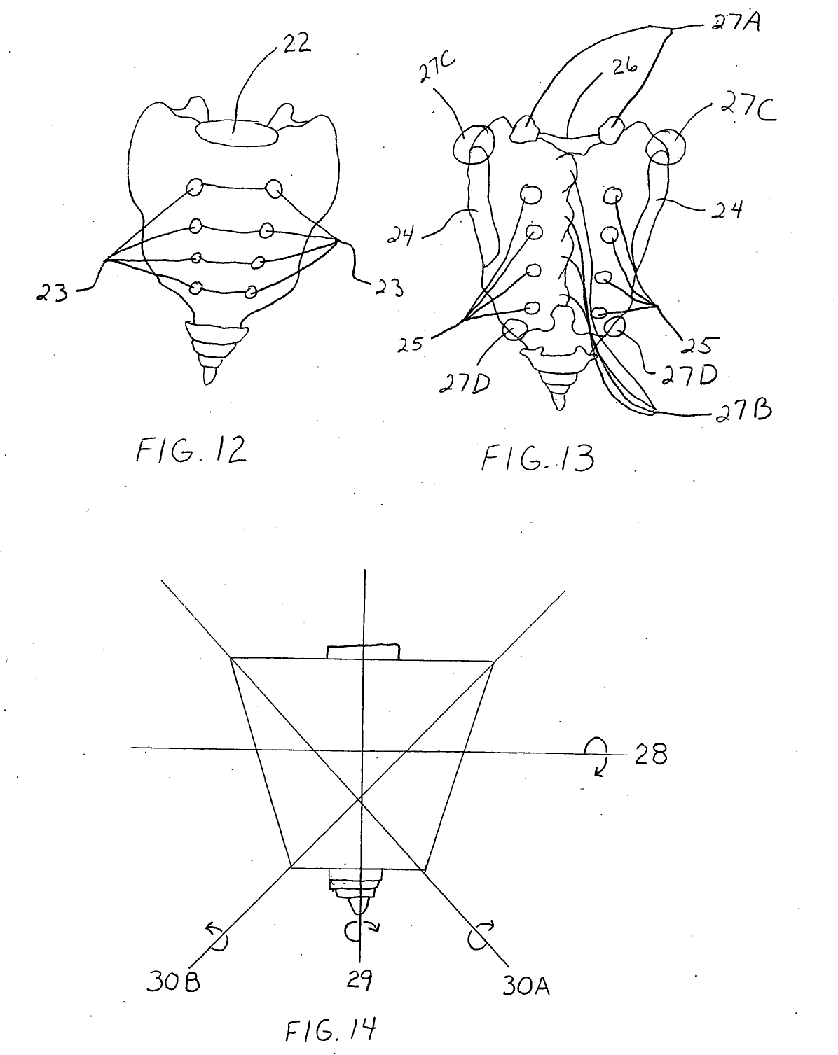

A front, or anterior, view of the human sacrum is shown in FIG. 12 and a back, or posterior, view is shown in FIG. 13. The lumbo-sacral articular surface 22,26 is shown superiorly. Nerve roots pass within the sacral canal and through the anterior sacral foramina 23. The posterior sacral foramina 25 are shown in FIG. 13. The sacroiliac joint articular surfaces 24, the median sacral crest, or sacral spinous processes, 27B, and the facets of the superior articular processes 27A are also shown in FIG. 13.

FIG. 14 is a representation of a human sacrum with the axis of sacral flexion and extension 28, rotation 29, and torsion 30A, 30B demonstrated by the strait lines and circular arrows.

The novelty of this invention is in the design of the sacral attachment FIG. 3. It is noted here and should be readily apparent to one skilled in the art that by simply removing or making detachable pegs 18,19,20,21 that the sacral attachment could function as a separate unit without the need for the base plate FIG. 1. Furthermore, the device as shown in FIG. 5 could function as a single unit with the sacral attachment permanently in place. The advantage and utility of the device as presented here will become apparent in the following discussion.

Patients with sacral dysfunctions resulting in low back, buttock, hip or leg pain should use the device fully assembled with the sacral attachment and rhomboid rubber comfort pads in place on the base plate as shown in FIG. 5. The patient should begin by lying on their back on a flat, firm surface such as a floor with their knees bent to a level of comfort and hips slightly flexed. The assembled device FIG. 5 should be positioned beneath the patient's bent knees flat on the floor with the sacral attachment FIG. 3 on top, the rounded front edge 2C adjacent to the patient's buttocks, and the back edge 2B facing the patients feet. The patient then gently raises their pelvis off the floor and grasps the base plate by the handles 7 and slides the entire device beneath their pelvis. The pelvis is then lowered onto the sacral attachment FIG. 3 and the sacral attachment and base plate are aligned so that the upper portion of the sacroiliac joints 27C rest upon the rhomboid rubber comfort pads 23 (in place on the rhomboid elevations 14,15). Both the upper portion of the sacrun at the sacroiliac joints 27C and a portion of the iliac bones should be in contact with the rhomboid rubber comfort pads 23 on top of the rhomboid elevations 14,15. The inferior lateral angles 27D of the sacrum should contact the rhomboid rubber comfort pads 22 on top of the rhomboid elevations 12,13. The median sacral crest 27B should project into the curved depressions 16B, 17B. As the patient's weight is lowered fully onto the sacral attachment, the sacral dysfunction, whether flexion, extension, rotation or torsion, will be corrected by the anterior displacement of the most posterior corners of the sacrum with subsequent posterior movement of the anteriorly displaced corners depending on the dysfunction. This motion will act to reseat, or square, the sacrum within the pelvis. Over-correction is prevented by the overlap of the rhomboid elevations 14,15 between the sacrum and iliac bones, which allows the sacrum only to move to a point in equilibrium with the iliac bones and the rest of the pelvis. By combining the sacral attachment with the wedge shaped main base plate FIG. 5 the benefit of lumbar lordosis reduction and sacral dysfunction correction are achieved simultaneously. Furthermore, the gentle gapping motion created between the fifth lumbar vertebrae and sacrum at the lumbo-sacral joint accentuates the correction of sacral misalignment, or dysfunction.

By creating a removable sacral attachment, the device can also be employed in the reduction of lumbar lordosis alone when used without the sacral attachment as shown in FIGS. 6 through 8. The device is positioned in the same manner as described above without the sacral attachment in place. The quadrilateral elevation 1A with quadrilateral rubber pad FIG. 2 in place would be positioned with the front edge 1B of the quadrilateral elevation 1A at the level of the lumbo-sacral junction 26, and the back edge 1C of the quadrilateral elevation 1A at the approximate level of the inferior lateral angle 27D of the sacrum. The patient then lowers their weight onto the quadrilateral elevation 1A and base plate. The slope of the device will act to displace the inferior lateral angles 27D anteriorly and cause the sacral base to move posteriorly. The entire pelvis will rotate and accentuate this movement and acts to reduce the lumbo-sacral angle and gently stretches the erector spinae muscles resulting in a flattening of the lumbar lordotic curve.

What is claimed is:

1. A therapeutic device comprising an overall wedge shaped, rigid base that is bilaterally concave with a central quadrilateral elevation.

2. A central quadrilateral elevation as in claim 1 maintains the same general slope as the wedge shaped base claimed in claim 1.

3. A comfort padding material covering the central quadrilateral elevation claimed in claim 1.

4. A rigid sacral attachment of the same overall dimensions as the central quadrilateral elevation claimed in claim 1, consisting of four smaller rhomboid elevations located in each corner of the sacral attachment.

5. A rigid sacral attachment as in claim 4 maintains the same general slope as the rigid base claimed in claim 1.

6. A comfort padding material covering each of the four smaller rhomboid elevations claimed in claim 4.
